(19) United States
(12) Patent Application Publication
(10) Pub. No.: US 2005/0014977 A1
(43) Pub. Date: Jan. 20, 2005

(54) Process for the carbonylation of epoxides

(76) Inventors: Eit Drent, Amsterdam (NL); Rene Ernst, Amsterdam (NL)

Correspondence Address: Jennifer D. Adamson;Shell Oil Company, Legal-Intellectual Property, P.O. Box 2463, Houston, TX, 77252-2463, US

(21) Appl. No.: 10/820,958

(22) Filed: Apr. 8, 2004

(30) Foreign Application Priority Data
Apr. 9, 2003 (EP) 03252260.9

(51) Int. Cl. C07D 487/22; C07C 27/02

(52) U.S. Cl. 568/451; 540/145

(57) ABSTRACT

The present invention pertains to a process for the carbonylation of an epoxide by reacting it with carbon monoxide in the presence of a catalyst system containing two components, wherein the first component is a source of one or more metals selected from the group consisting of cobalt, ruthenium and rhodium, and the second component is a coordination complex of a tetrapyrrole compound with one or more of the metals belonging to the group consisting of groups IIIA and IIIB of the periodic system, lanthanides and actinides. The present invention also pertains a process for the preparation of a catalyst system.

### FIELD OF THE INVENTION

The present invention pertains to a process for the carbonylation of epoxides, to a catalyst system suitable for this process, and to a process for the preparation of the catalyst system.

### BACKGROUND OF THE INVENTION

Generally, carbonylation is understood as the insertion of a carbonyl or a carbonyl group into an organic compound. For instance, the reaction of an epoxide compound with carbon monoxide in the presence of a catalyst comprising a metal selected from group VIII of the periodic system (as defined on page 1-11 of the CRC Handbook of Chemistry and Physics, 72nd Edition, 1991) is such a carbonylation reaction. Processes for the carbonylation of epoxides may be found in the literature. EP-A-0,577,206, for instance, describes the carbonylation of epoxides to obtain β-lactones or β-hydroxycarboxylic acid derivatives of these lactones in the presence of a catalyst system comprising a source of cobalt and hydroxy substituted pyridine. Although it proceeds smoothly with ethylene oxide, this process does not give satisfying results with substituted epoxides, such as propylene oxide. A further problem with the carbonylation of propylene oxide in this process is that instead of the desired β-butyrolactone the process may yield, partially or completely, a polyester product, as described in J. Am. Chem. Soc. 124, 2002, 5646-5647. An improved catalyst system for the carbonylation of epoxide substrates having different substituents, the catalyst comprising several cationic Lewis-acid coordination complexes and a source of cobalt has been described in J. Am. Chem. Soc. 124, 2002, 1174-1175. Although the metal complexes and catalyst systems described in this document allow conversion of different epoxide substrates to the corresponding monomeric β-lactone products, yields and selectivity are typically low. Therefore, it would be desirable to have a catalyst system with a higher catalytic activity. It would furthermore be desirable to have a catalyst system and a process that would yield, solely or mainly, a monomeric product.

### SUMMARY OF THE INVENTION

The present invention is directed to a process for the carbonylation of an epoxide by reacting it with carbon monoxide in the presence of a catalyst system containing two components, wherein the first component is a source of one or more metals selected from the group consisting of cobalt, ruthenium and rhodium, and the second component is a coordination complex of a tetrapyrrole compound with one or more of the metals belonging to the group consisting of groups IIIA and IIIB of the periodic system, lanthanides and actinides.

### DETAILED DESCRIPTION OF THE INVENTION

There has now been found a novel catalyst system which is highly effective in the carbonylation of epoxides and offers the advantage of a significantly higher turnover (defined as mol product/mol catalyst employed) to the corresponding β-lactones, without the formation of significant amounts of undesired by-products and polymerization products.

Within the context of the present invention, carbonylation of epoxides represents the insertion of a carbonyl into an oxirane moiety under formation of a 2-oxetanone (β-lactone) structure.

The first component of the catalyst system according to the present invention is a source of one or more metals selected from the group consisting of cobalt, ruthenium and rhodium. These metals are active in this reaction. The choice of metal depends on the circumstances, such as epoxide substrate and desired products. A preferred metal for use in the first component is cobalt due to the proven high catalytic activity and good availability of suitable starting materials. Particularly preferred due to the ease and safety of their preparation are the metal tetracarbonyl salts. Accordingly, the present invention pertains to the process for carbonylation of an epoxide, wherein the first component is a metal tetracarbonyl. A preferred first component of the catalyst system is a cobalt tetracarbonyl, as for instance described by Edgell and Lyford in Inorganic Chemistry, Vol. 9, No. 8, 1970, pages 1932 to 1933.

The second component of the catalyst system according to the present invention is a coordination complex of a metal selected from the group consisting of groups IIIA and IIIB of the periodic system, lanthanides and actinides with a tetrapyrrole compound. Without wishing to be bound to any particular theory, it is believed that the positively charged metal/tetrapyrrole ligand coordination complex acts as a Lewis acid by coordinating the epoxide, thereby promoting the insertion of the carbon monoxide into the thus activated epoxide bond. The oxidation state of the metal atoms during the carbonylation of epoxides may vary substantially as well as change during the course of the reaction. Preferably, these metals form, in oxidation state +III, a stable, positively charged coordination complex with the tetrapyrrole compound, which acts as a tetradentate dianionic ligand. Therefore, the metal preferably resides in an oxidation state of +III, thereby providing two free valences which may act as binding sites to the tetrapyrrole ligand, whereas the third free valence acts as a positive charge whereby the metal coordination complex acts as counter-ion to the anionic cobalt carbonyl complex. Preferred metals are aluminium, indium, gallium, scandium, ytterbium, lanthanum, cerium and samarium. Of these, aluminium is the most preferred due to its commercial availability and to the high stability of aluminium (III) complexes. Accordingly, the present invention preferably is directed to a process, wherein at least part of the metal in the second component is aluminium.

Tetrapyrrole compounds in the second component are members of a class of compounds whose molecules have four pyrrole rings which can act as dianionic tetradentate ligands with metal atoms. Common arrangements of the pyrrole rings may be macrocyclic or linear. Preferred are the tetrapyrrole macrocyclic rings commonly denominated as porphyrines. These porphyrines contain a fundamental skeleton of four pyrrole nuclei united through the α-positions by four methine groups to form a macrocyclic structure. The porphyrine ligands suitable for use according to the present process may bear one or more alkyl substituents such as methyl ethyl, n- and isopropyl and butyl, aryl substituents such as optionally substituted phenyl substituents, and substituents comprising heteroatoms at any position other than the nitrogen atoms of the pyrrole rings. One or more of these substituents other than hydrogen atoms may be present at the positions 2, 3, 5, 7, 8, 10, 12, 13, 15, 17, 18 and 20 of the porphyrine nucleus (as defined by the IUPAC in the recommendations 1978, Pure Appl. Chem. 51, 2251-2304, 1979). Accordingly, the present invention preferably is directed to the subject process wherein the tetrapyrrole compound is a porphyrine compound. More preferred porphyrines include tetraarylporphyrines such as (5, 10, 15, 20-tetraphenyl)porphyrine, tetrakis-(4-methoxyphenyl)-porphyrine, tetrakis-(2-methoxyphenyl)-porphyrine, tetrakis-(2-chlorophenyl)-porphyrine, tetrakis-(2-hydroxyphenyl)-porphyrine and tetrakis-(2,4-dimethoxyphenyl)-porphyrine. Other suitable tetrapyrrole ligands are di-benzoporphyrine and tetra-benzoporphyrine and cyclopentaporphyrine, and the naturally occurring members of the porphyrine family. Most preferred due to the commercial availability and proven efficacy is (5,10,15,20-tetraphenyl)porphyrine.

The oxirane ring of the epoxide reactant in the subject process may be substituted with alkyl and aryl groups, as for instance in propylene oxide or styrene oxide. The epoxide reactant may also bear other functional groups such as for instance in epichlorohydrin, or it may be part of a saturated cyclic structure such as epoxidized cyclohexene. However, more suitable, due to a fast and selective reaction, are optionally substituted 1,2-epoxyalkanes. Representative 1,2-epoxides include ethylene oxide, propylene oxide, butylene oxide, styrene oxide, 1,2-epoxyhexane and 1,2-epoxyoctane of which ethylene oxide and propylene oxide are most suitable.

The present invention also is directed to a process for the preparation of the catalyst system. Suitable methods include the stepwise preparation in-situ or prior to the epoxidation process, and the in-situ self-assembly method. The preferred process for preparing the catalyst system is the stepwise preparation of the catalyst. Accordingly, the present invention preferably is directed to a process for the preparation of a cobalt containing catalyst system suitable for the carbonylation of epoxides, which process comprises the steps of:
(a) reacting a source of at least one metal selected from the group consisting of groups IIIA and IIIB of the periodic system, lanthanides and actinides with a tetrapyrrole compound, and
(b) reacting the product of step (a) with a source of at least one metal selected from the group consisting of cobalt, ruthenium and rhodium to obtain the catalyst complex.

Step (a) of the catalyst preparation process is the synthesis of the metal-ligand coordination complex. This may be achieved by bringing a suitable metal source in contact with the selected tetrapyrrole ligand, for instance by using the method as described by Aida and Inoue in J. Am. Chem. Soc. 1983, 105, 1304-1309. The metal ligand complex formed may be directly converted further, or isolated at this stage. Without wishing to be bound to any particular theory, it is believed that in the metal coordination complex, the metal ion is coordinated to the tetrapyrrole as a tetradentate dianionic ligand, having one or more additional axial ligands. Preferably due to the proven high reactivity the source of a metal of step (a) comprises aluminium. Even more preferably, the tetrapyrrole compound is a porphyrine compound. Accordingly the present invention preferably is directed to a process for the preparation of a catalyst system, wherein the metal in step (a) is aluminium, and to a system wherein the tetrapyrrole compound is a porphyrine compound.

In step (b), the metal-ligand coordination complex of step (a) is reacted with a source of metal selected from the group consisting of cobalt, ruthenium and rhodium. This source of a metal may be introduced into step (b) in any form that may be converted during step (b) into a suitable anionic metal carbonyl species. The source of metal preferably comprises cobalt, more preferably being introduced as alkali metal tetracarbonyl cobalt salt prepared prior to step (a). Accordingly, the present invention is directed to a process for the preparation of a catalyst system, wherein the source of metal of step (b) is a cobalt tetracarbonyl sodium salt.

The conditions at which the catalyst system is prepared in steps (a) and (b) respectively are not critical. Temperature and pressure may vary within the range of from minus 70° C. to plus 150° C., more preferably in the range of from 0° C. to 90° C., and most preferably in the range of from 15° C. to 40° C. At this point, optionally, the catalyst system may be isolated. Also within the scope of the invention is a self-assembly method, wherein the catalyst components are brought together at the same time, optionally under carbon monoxide pressure. Selection of suitable conditions lies within the capability of a person skilled in the field of organometal complexes.

The molar ratio of the second catalyst component (i.e. the metal coordination complex) to the first catalyst component may vary within relatively broad ranges. Suitably, the molar ratio varies from 4:1 to 1:4, preferably from 3:1 to 1:3, and most preferably from 2:1 to 1:2.

The catalyst system according to the present invention is believed to comprise a novel bimetallic catalyst system.

Accordingly, the present invention preferably also pertains to the catalyst system obtainable by the above-described process, and to its use for the carbonylation of an epoxide.

The subject process has the further advantage that it may be performed neat, i.e. in the absence of additional solvent if the substrate is liquid under the conditions of the reaction. This facilitates work-up and purification procedures. However, any suitable solvent may be employed, in particular during the start-up phase of the reaction, or during the in-situ preparation of the catalyst system in the reaction vessel.

A suitable solvent is inert in the carbonylation reaction, meaning that it is not consumed during the course of the reaction. Suitable solvents for the process according to the present invention will sollubilise the feeds during the course of the reaction. Such solvents include cyclic or linear ethers of diols such as tetrahydrofurane (thf) and alkyl substituted furans, or diethylene glycol dimethyl ether (diglyme) due to their high solvency. It has, however, been observed that the reaction can proceed more smoothly and faster in absence of additional solvent. Therefore, the present process more preferably is performed in liquid product and in the absence of additional solvents.

In a different embodiment of the present invention, the reaction is performed in the presence of solvents having active hydrogen atoms, for instance, alkanols. Although these solvents do not interfere with the carbonylation reaction, they can further react under the conditions of the carbonylation reaction with the initially formed β-lactone product to produce β-hydroxy-compound esters and/or derivatives thereof, such as α,β-unsaturated compounds.

The optimum ratio of epoxide in the feed to catalyst complex will in part depend upon the particular complex employed. Preferably, the molar ratio of epoxide to the first metal can be in the range of from 10² to 10⁷, and more preferably in the range of from 2*10² to 10⁶. [0021] The carbonylation is conveniently conducted under conditions of elevated temperature. Although the reaction does proceed at lower temperatures, good results are achieved at temperatures above room temperature. Accordingly, reaction temperatures may preferably range from 30° C. to 150° C., more preferably from 50° C. to 125° C., and most preferably from 60° C. to 110° C. At lower temperature, the reaction may be unduly retarded, whereas higher temperatures may induce the formation of secondary derivatives such as polymeric material.

The present process further requires elevated pressure, which is preferably achieved by pressurizing with carbon monoxide, and/or with a gas mixture comprising carbon monoxide and gases such as for instance nitrogen or hydrogen are suitable. Preferably, the molar ratio of carbon monoxide to the other gases in the mixture, when present, is within a range of from 0.1 to 10, more preferably from 1 to 10.

Typical total pressures are below 150*10⁵ N/m² (150 bar), as higher pressure would involve complex and cost-intensive equipment. The process is thus preferably performed at a total pressure in the range of from 30*10⁵ N/m² to 150*10⁵ N/m², more preferably in the range of from 40*10⁵ N/m² to 120*10⁵ N/m², again more preferably of from 50*10⁵ N/m² to 100*10⁵ N/m², and most preferably of from 60*10⁵ N/m² to 90*10⁵ N/m².

Although the temperature and pressure for the carbonylation are not critical and may thus vary within wide limits, it is an advantageous feature of the invention that the reaction can be conducted at relatively mild conditions.

The process according to the present invention will be further illustrated by reference to the following non-limiting examples.

### EXAMPLE 1

A 250 ml Hastelloy C reactor (Hastelloy C is a registered trademark of Haynes International Inc.) equipped with a magnetic stirrer, a heating unit and an inlet was charged with 50 ml diethylene glycol dimethyl ether (diglyme) and the catalyst precursors (97 mg (0.5 mmol) of Na[Co(CO)₄)], 337 mg (0.5 mmol) of (5, 10, 15, 20-tetraphenyl)porphyrine aluminium chloride), then purged with nitrogen, and finally pressurized with carbon monoxide (CO) to a pressure of 10*10⁵ N/m². Then 15 ml (307 mmol) of ethylene oxide (EO) were pumped into the reactor. The pressure in the reactor was then increased further with CO to 50*10⁵ N/m², and further with hydrogen to the final pressure of 70*10⁵ N/m². Then the reactor was heated to 70° C. and kept at this temperature for a period of 10 hours under vigorous stirring. At the end of the reaction, the gas consumption was determined as 20*10⁵ N/m².

The conversion and turnover number of ethylene oxide to β-propiolactone (TON) were determined by GC-analysis. The conversion of EO is expressed in (mol)%, and is based on the molar amount of converted EO divided by the molar amount of EO supplied times 100%. The amount of lactone formed was calculated from the ratio of remaining EO to obtained lactone. The turnover number (TON) of ethylene oxide to β-propiolactone is defined as mol lactone obtained/mol catalyst employed. The reaction had proceeded with a turnover number (TON) of 389 at a conversion of 63%.

### EXAMPLE 2

Example 1 was repeated, however, using propylene oxide as substrate. The TON and conversion of ethylene oxide to β-propiolactone were determined by GC- and ¹H-NMR-analysis as 358 at 49% conversion.

### Comparative Example 1

Example 1 was repeated, however, employing the cobalt-containing aluminium-salen catalyst system as described in J. Am. Chem. Soc. 124, 2002, 5646-5647, and ethylene oxide as substrate. The resulting TON was determined by GC-analysis as 98 at 32% conversion.

### Comparative Example 2

Comparative example 1 was repeated, however, using propylene oxide as substrate. The TON was determined by GC- and ¹H-NMR-analysis as 123 at 49% conversion.

The much higher conversion achieved with the novel catalyst system according to the present invention with respect to alternative catalyst systems shows that the process for the carbonylation of epoxides according to the present invention represents a large improvement over the process known in the art.

1. A process for the carbonylation of an epoxide comprising reacting the epoxide with carbon monoxide in the presence of a catalyst system comprising two components, wherein the first component is a source of one or more metals selected from the group consisting of cobalt, ruthenium and rhodium, and the second component is a coordination complex of a tetrapyrrole compound with one or more of the metals belonging to the group consisting of groups IIIA and IIIB of the periodic system, lanthanides and actinides.

2. The process of claim 1, wherein the metal of the first component is cobalt.

3. The process of claim 2, wherein the first component is a metal tetracarbonyl.

4. The process of claim 2, wherein the metal of the second component is aluminium.

5. The process of claim 2, wherein the tetrapyrrole compound is a porphyrine compound.

6. The process of claim 2, wherein the epoxide is selected from the group consisting of ethylene oxide and propylene oxide.

7. The process of claim 2, wherein the carbonylation is conducted in the presence of a solvent having an active hydrogen atom.

8. The process of claim 1, wherein the first component is a metal tetracarbonyl.

9. The process of claim 1, wherein the metal of the second component is aluminium.

10. The process of claim 1, wherein the tetrapyrrole compound is a porphyrine compound.

11. The process of claim 1, wherein the epoxide is selected from the group consisting of ethylene oxide and propylene oxide.

12. The process of claim 1, wherein the carbonylation is conducted in the presence of a solvent having an active hydrogen atom.

13. A process for the preparation of a catalyst system suitable for the carbonylation of epoxides, which process comprises the steps of:
(a) reacting a source of at least one metal selected from the group consisting of groups IIIA and IIIB of the periodic system, lanthanides and actinides with a tetrapyrrole compound; and,
(b) reacting the product of step (a) with a source of at least one metal selected from the group of cobalt, ruthenium and rhodium.

14. The process of claim 13, wherein the metal of step (a) is aluminium, and wherein the tetrapyrrole compound is a porphyrine compound.

15. The process of claim 14, wherein the source of metal of step (b) is a cobalt tetracarbonyl sodium salt.

16. The process of claim 13, wherein the source of metal of step (b) is a cobalt tetracarbonyl sodium salt.
